(19) United States
(12) Patent Application Publication
(10) Pub. No.: US 2026/0276496 A1
(43) Pub. Date: Sep. 17, 2026

(54) SYSTEM FOR AND METHOD OF MONITORING A GAS COMPONENT

(71) Applicant: POST HARVEST IP PTY LTD, Sydney (AU)

(72) Inventors: Jason Robert POTAS, Sydney (AU); Daniel JAJCEVIC, Sydney (AU); Jeremy Anthony RODARELLIS, Sydney (AU); Alexander HATZIMIHALI, Sydney (AU); Joshua DUNFORD, Sydney (AU); Jonathan SHANNON, Sydney (AU); Michael POTAS, Sydney (AU)

(73) Assignee: POST HARVEST IP PTY LTD, Sydney (AU)

(21) Appl. No.: 19/399,507

(22) Filed: Nov. 24, 2025

Related U.S. Application Data
(63) Continuation-in-part of application No. PCT/AU2024/050534, filed on May 23, 2024.

(30) Foreign Application Priority Data
May 24, 2023 (AU) 2023901621

(51) Int. Cl. G01N 1/40; G01N 33/00

(52) U.S. Cl. CPC G01N 1/4005; G01N 1/4022; G01N 2001/4016; G01N 1/405; G01N 33/0047

(57) ABSTRACT

Described is a method (900) of measuring a small concentration of a target gas component in an environmental gas sample by increasing the small concentration to a larger measurable concentration. The method comprises isolating and concentrating (930) the target gas component using a concentrator (106), by capturing the target gas component and releasing the captured target gas component to a sensor (108). Isolating the target gas component comprises increasing a concentration of the target gas component so that the concentration of the target gas component is measurable by the sensor. The method also comprises measuring (940), using the sensor (108), the captured target gas component concentration. Before isolating the target gas component, the method comprises heating the concentrator to release a baseline gas, and measuring, using the sensor (108), a baseline gas component concentration (920). After measuring the captured target gas component concentration, the method comprises determining (950), with a processor (112, 202), an accurate target gas component concentration by comparing the baseline gas component concentration and the measured target gas component concentration.

### CROSS-REFERENCE TO RELATED APPLICATION

This application is a continuation in part of International Application No. PCT/AU2024/050534, filed May 23, 2024, claiming priority to Australian Provisional Patent Application No. 2023901621, filed May 24, 2023, contents of which are hereby incorporated by reference in their entirety.

### TECHNICAL FIELD

The present disclosure broadly relates to the monitoring of gas components of air and, more particularly, to a system for, and a method of monitoring ethylene.

### BACKGROUND

Sensor systems are used to monitor fresh produce during shipment, storage and ripening. Certain aerosolised or gaseous compounds issue from ripening produce. Reaching a threshold level, or changing amounts of one or more of the compounds provides an indicator of different stages of the ripening process, or can provide indicators of certain anomalies in the ripening process.

Ethylene is an example of a gas released by some fruit and vegetables as they ripen. Ethylene sensors can be used to monitor the ripening process of stored or transported produce by measuring the ethylene concentration of the air in the confined space. This can be difficult not only because the ethylene concentration can be very small (less than 1 ppm), but also because the accuracy of the sensors can be affected by other gas components in the air such as carbon monoxide.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Described herein systems and methods for measuring gas samples that are otherwise too low to detect by commercially available gas sensors. The sensing mechanism works by isolating and concentrating the gas component, thereby amplifying the gas signal to a level that can be accurately measured by a gas sensor that would otherwise lack sensitivity. The system includes an ethylene sensor (e.g. a commercially available electrochemical sensor, such as Membrapor CA-10 sensor, an absorption spectroscopy sensor, etc), a gas concentrator unit, and a temperature controlled heating element. The gas sensing works by passing a fixed quantity of gas though the concentrating unit when the unit is relatively cold. During this mode, the gas is sequestered inside the concentrator unit. The measuring mode is evoked by heating the concentrator to a level that releases all the stored gas at once, so that its concentration is much higher than before. The concentrated gas then passes over the ethylene gas sensor at a concentration that can be measured reliably. The effect is a reading from the ethylene sensor that would otherwise not have the sensitivity to read anything.

As used herein, references to a “small concentration” or “low concentration” of ethylene refers to ethylene concentrations of 1 to 2 ppm or less. References to a “measurable concentration” of ethylene refers to concentrations of about 10 ppm or more.

In one aspect there is provided a method of measuring a small concentration of a target gas component in an environmental gas sample by increasing the small concentration to a larger measurable concentration, the method comprising: isolating and concentrating, using a concentrator, the target gas component by capturing the target gas component and releasing the captured target gas component to a sensor; and measuring, using the sensor, the captured target gas component concentration, wherein isolating and concentrating the target gas component comprises increasing a concentration of the target gas component so that the concentration of the target gas component is measurable by the sensor.

The method also comprises, before isolating the target gas component, heating the concentrator to release a baseline gas, and measuring, using the sensor, a baseline gas component concentration; and after measuring the captured target gas component concentration, determining, with a processor, an accurate target gas component concentration by comparing the baseline gas component concentration and the measured target gas component concentration.

The method may comprise removing, using a filter, an unwanted gas component by capturing the unwanted gas component and releasing the captured unwanted gas component to an exhaust; effecting a reverse airflow to remove the unwanted gas component (e.g. moisture) without the unwanted gas contacting the concentrator; and effecting a forward airflow to release the target gas component toward the sensor.

The method may further comprise, before the isolating and concentrating, a bypass cycle comprising: measuring, using the sensor, a concentration of the target gas component in the environmental gas sample and, if the concentration is above a threshold, then directing airflow directly to the sensor without concentrating it, so that the isolating and concentrating step occurs following and dependent on the bypass cycle.

In another aspect there is provided a system for monitoring a gas component, the system comprising: a gas input in communication with a gas output via: a concentrator configured for capturing and then releasing the gas component from an airflow through the system; and a sensor system configured to sense a concentration of the released gas component; and a controller configured to control operation of the concentrator to: isolate and concentrate the target gas component by capturing the target gas component and releasing the captured target gas component to the sensor system, thereby increasing a concentration of the target gas component so that the concentration of the target gas component is measurable by the sensor system.

The system further comprises a processor configured to determining an accurate target gas component concentration by comparing the baseline gas component concentration and the measured target gas component concentration.

The controller may be configured to control operation of the concentrator to effect heating of the concentrator to release a baseline gas so that the sensor measures a baseline gas component concentration.

The system may further comprise a first filter for removing an unwanted gas component, wherein the first filter comprises a first aluminosilicate compound having a first pore size. The concentrator may comprise a second aluminosilicate compound comprising a second pore size, wherein the second pore size is larger than the first pore size.

The first aluminosilicate compound may comprise a zeolite with a pore size of about 3 Å, and the second aluminosilicate compound may comprises a zeolite with a pore size of about 4 Å.

The system also includes a preconditioner, which is use to condition the gas before it enters the concentrator. The main role of the preconditioner is to remove water in the gas so that it is not concentrated along with the ethylene. The concentrator and the preconditioner both contain zeolites, which are used to adsorb and release gases under different temperatures. Other key components may include a pump for pushing the gas through the system, and a controller to control the components.

The systems and methods described herein can be used to measure ethylene levels to monitor the ripeness of produce. Advantageously, the systems have been shown to accurately and consistently detect low concentration levels of ethylene (less than 1 ppm).

Throughout this specification the word “comprise” or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the disclosure are now described by way of example with reference to the accompanying drawings in which:

FIG. 1 is a schematic diagram of a system for monitoring a gas component.

FIG. 2 is a schematic representation of a controller forming part of the system in FIG. 1.

FIG. 3 is a schematic representation of a control system executed by the controller.

FIG. 4 is a schematic representation of an example embodiment of an ethylene monitoring system.

FIG. 5 illustrates graphs of sensor readings from the system of FIG. 4.

FIG. 6A illustrates an example of a zeolite gas filter.

FIG. 6B is a diagram of the ethylene molecule size range compared to the pore sizes of zeolites.

FIG. 7 illustrates the operation of a sensing cycle.

FIG. 8 illustrates one embodiment of a method of monitoring a gas component.

FIG. 9A illustrates one embodiment of a method of monitoring a gas component.

FIG. 9B illustrates a method of isolating and concentrating a gas component.

FIG. 10 is a schematic representation of the operation of the gas component monitoring systems.

FIG. 11A is a schematic representation of the system architecture.

FIG. 11B illustrates a standard measurement cycle with respect to the system architecture of FIG. 11A.

FIG. 11C illustrates a preconditioner cleaning cycle with respect to the system architecture of FIG. 11A.

FIG. 11D illustrates a bypass cycle with respect to the system architecture of FIG. 11A.

FIG. 12 is a graph of ethylene measurements with respect to different cycle times.

FIG. 13 is a graph of ethylene measurements over the first and the second heat cycle for different ethylene concentrations.

FIG. 14A is a graphical representation of the measured ethylene concentration.

FIG. 14B is a graphical representation of a first and a second measurement of the ethylene concentration shown in FIG. 14A.

### DETAILED DESCRIPTION

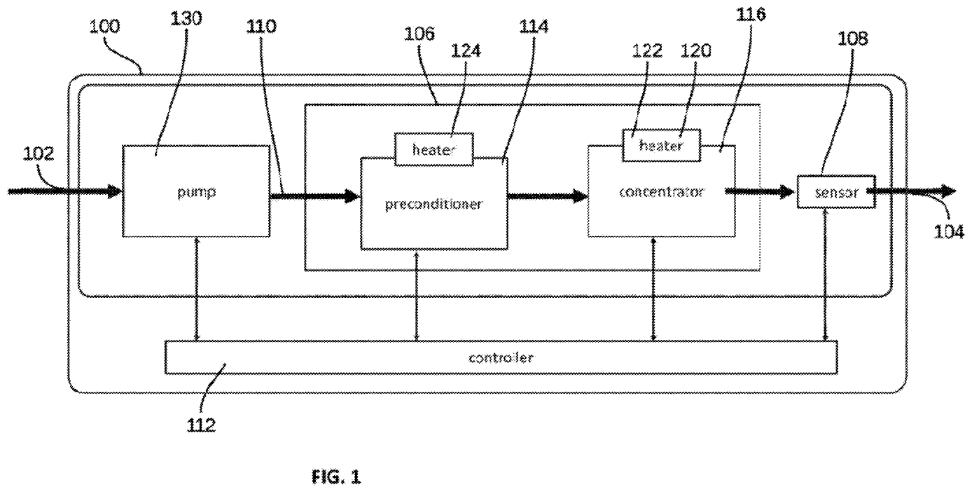

FIG. 1 of the drawings shows a system 100 for monitoring a gas component, such as ethylene, in an environmental gas sample (for example, in a container holding fresh produce such as fruit or vegetables). The system comprises a gas input 102 in communication with a gas output 104 via a filter and concentrator system 106 and a sensor system 108. The filter and concentrator system 106 is adapted for isolating and concentrating the gas component (e.g., ethylene) from an airflow 110 through the system 100, thereby increasing the concentration of the gas component from a small concentration to a larger measurable concentration. The sensor system 108 is adapted to sense the larger measurable concentration of the isolated gas component. The system 100 also includes a controller 112 configured to control operation of the filter and concentrator system 106, as well as other components of the system 100. The system 100 includes a pump system 130 that comprises one or more pumps that effect airflow through the system 100. In alternative embodiments other pumping mechanisms may be used, for example compressed air.

The sensor system 108 comprises an ethylene sensor, for example an electrochemical sensor (e.g., Membrapor CA-10), an infrared sensor, and/or an absorption spectroscopy sensor.

The filter and concentrator system 106 has a first filter in the form of a preconditioner 114 for removing an unwanted gas component, for example moisture in the form of water molecules. The system also has a concentrator 116 that isolates the gas component and increases the concentration of the gas component.

Some embodiments may not include a preconditioner, or may be configured to selectively bypass the use of a preconditioner. This may be suitable for certain sensor types, for example infrared sensing that is not significantly or detrimentally affected by unwanted gasses, for example by humidity.

In normal operation of the system, air flows through the preconditioner first then through the concentrator. The preconditioner is used to collect water in order to avoid humidity affecting the sensor readings; this is useful because humidity can be as high as 95% in cool rooms. The preconditioner also collects other particles smaller than 3 Angstrom in pores at <~50-100 degrees Celsius, therefore advantageously removes at least some unwanted gasses. When heated to >~100-200 degrees, the pores of the filtering beads release the gas.

The concentrator is used to collect the ethylene for measurement. The concentrator collects everything smaller than about 4 Angstrom when the pores are at <~50-150 degrees Celsius, and then when heated to >~150-300 degrees, the beads release those captured gas molecules again.

The lower and higher temperature ranges that the heaters for the preconditioner and concentrator operate at may vary depending on the filter elements used (i.e. the specific beads), environmental conditions (e.g. temperature and pressure), and molecules targeted.

In alternative embodiments the lower and higher temperature ranges may be reduced by including a pressure control system, for example a piston.

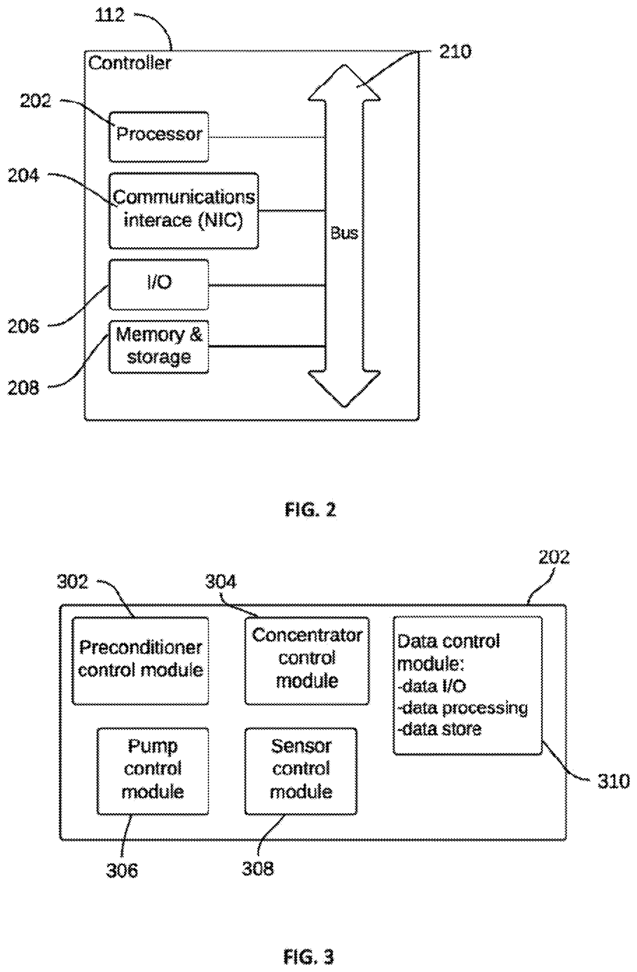

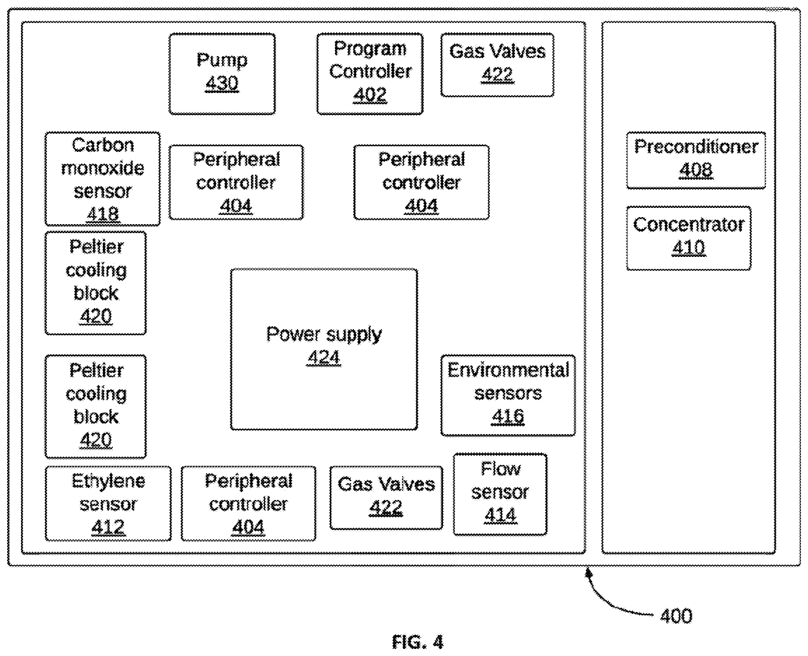

The controller 112 may be implemented using any suitable computing technology, such as a microcontroller (e.g., an Arduino™ or Raspberry Pi™), a microprocessor, a DSP, FPGA, etc. An exemplary configuration of a centralised architecture is shown in FIG. 2 of the drawings, in which the controller 112 includes various components including a processor 202, communications interface (NIC) 204, an input/output (I/O) interface 206, and memory and/or storage 208, connected via a bus 210. The memory 208 includes various instructions that, when executed by the processor 202, operate a number of control modules 300. As illustrated in FIG. 3 of the drawings, the control modules 300 may include one or more of: a preconditioner control module 302, a concentrator control module 304, a pump control module 306, a sensor control module 308, and a data control module 310. In some embodiments, the control modules may also include one or more heater control modules.

It will be understood that the control functions effected by the controller 112 may be implemented in various formats, using various combinations of hardware and software to control operation of the system 100 as described herein.

FIG. 4 of the drawings shows an embodiment of a monitoring system 400 with a distributed controller architecture with a central program controller 402 (e.g., a Raspberry Pi™ microcontroller) and various peripheral controllers 404 (e.g. Arduino™ microcontrollers). The program controller 402 is responsible for controlling the overall operation of the system 400, and specifically coordinating operation of the various components, whereas the peripheral controllers 404 are each associated with one or more subsystems such as the pump 430, the preconditioner 408, the concentrator 410, etc. As can be seen, in addition to the ethylene sensor 412, the system 400 has a number of additional sensors including a flow sensor 414, environmental sensors 416, and a carbon monoxide sensor 418, with Peltier cooling blocks 420 associated with one or more of these sensors. Gas valves 422 are provided to direct gas flow around the system. The system 400 also includes a power supply 424 that manages the power provided to each of the system's components.

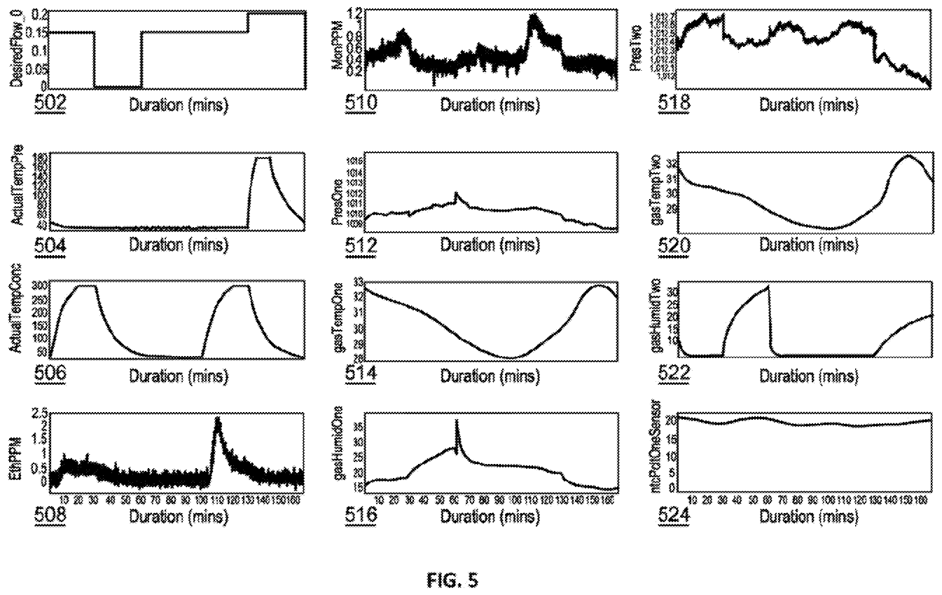

As shown in FIG. 5 of the drawings, the flow sensor 414 measures flow rates. In some embodiments the flow rates are controlled to be a target flow rate 502. Also illustrated in FIG. 5, temperature sensors provide the preconditioner temperature 504, the concentrator temperature 506, the inlet gas temperature 514, and the gas temperature after the preconditioner 520. Temperature sensors may also be used to monitor the temperature of the ethylene sensor block, the carbon monoxide sensor block, and/or the Peltier cooling block 524. The ethylene sensor 412 provides ethylene measurements 508. Some embodiments may include a carbon monoxide sensor 418 that provides carbon monoxide measurements 510, and humidity sensors that measure the gas humidity at the inlet 516 and after the preconditioner 522. Some embodiments may also include pressure sensors are used to monitor the gas pressure at the inlet 512 and after the preconditioner 518.

As illustrated in FIG. 1 of the drawings, the filter and concentrator system 106 comprises at least one controllable heating element 120 because the filter and concentrator system 106 includes components that are temperature dependent. Specifically, the preconditioner 114 and/or the concentrator 116 include a molecular sieve that traps molecules of a certain size whilst letting others through. In some embodiments, both the preconditioner 114 and the concentrator 116 include a molecular sieve in the form of an aluminosilicate compound such as zeolites. Typically, a different molecular sieve would be used in the preconditioner 114 and the concentrator 116, for example by using two different zeolites, so that molecules of differing sizes are targeted by the two molecular sieves.

Zeolites are microporous, crystalline aluminosilicate materials that mainly consist of silicon, aluminium, oxygen. Zeolites have different pore sizes, and the pore size governs their physical properties and abilities to separate molecules because only molecules of certain sizes and shapes are targeted by certain pore sizes.

For the system 100,400 described herein, the first filter comprises a molecular sieve with a first pore size. The concentrator comprises a molecular sieve with a second pore size, the first pore size being smaller than the second pore size. In this way, the first filter targets a different type of molecule than the concentrator.

In other embodiments, the molecular sieves of the filters may comprise one or more of: activated carbon, microporous silica, polysaccharides, and polymers having pore sizes suitable for filtering relevant molecules.

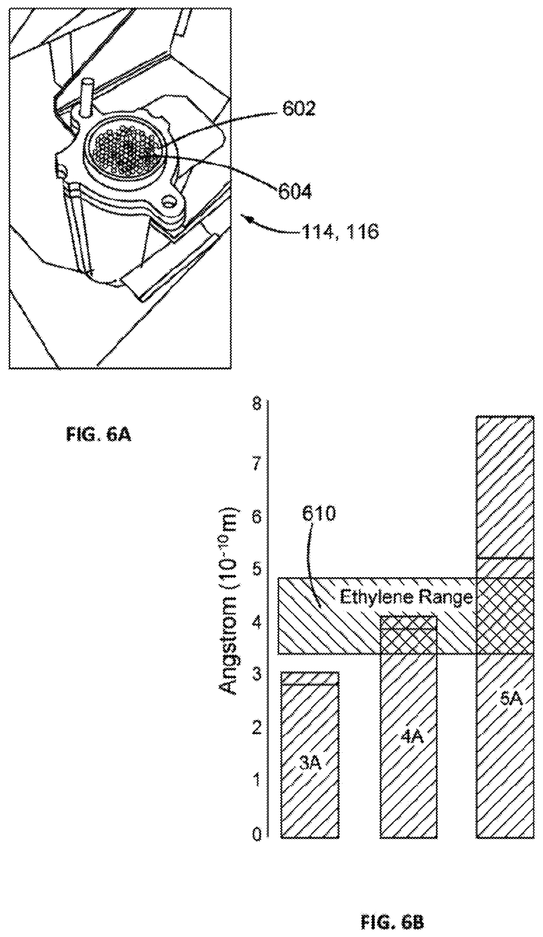

FIG. 6A of the drawings illustrates an example of a filtering component that comprises an outer body 602 defining an inner chamber holding a plurality of zeolites 604. Such filtering components may be used as a component for the filter and/or for the concentrator.

Water molecules are in the order of 2.8 Å, while ethylene is in the order of about 3.6 Å (3.28×4.18×4.84 Å³). Ethylene molecules have a trigonal planar shape, therefore they can be trapped and captured in certain orientations. Referring to FIG. 6B of the drawings, zeolites have a pore size range which is somewhat normally distributed. The figure shows these ranges for each zeolite and the ethylene dimensions (longest and shortest axes) for comparison. FIG. 6B shows that 4 Å will select for the shorter axis of ethylene, and 5 Å will adsorb ethylene in any orientation. The first 3 Å zeolites capture molecules sized 3 Å and smaller, but any larger molecules, such as ethylene, will not be trapped and pass over the crystalline structure. The second 4 Å zeolites capture molecules sized about 4.2 Å and smaller. The third 5 Å zeolites capture molecules sized around 5 Å and smaller, although there is variability in this type of zeolite, making it possible to capture molecules with sizes up to 8 Å. As illustrated, the ethylene size range 610 overlaps the 4 Å and 5 Å, although the inventors have found that the 4 Å provide effective results for the purpose of monitoring ripening produce.

The first pore size is between 2 and 4 Å, for example between about 2.8 and 3 Å, thus being suitable to capture water molecules so as to reduce moisture in the air. One example of a suitable zeolite for the first filter is the Sigma-Aldrich™ Molecular Sieve UOP Type 3 Å with linear formula KₙNa₁₂₋ₙ[(AlO₂)₁₂(SiO₂)₁₂]×H₂O.

The second pore size is between 3 and 5 Å, for example between about 3.8 and 4.2 Å, thus being suitable to capture ethylene from the airflow through the system 100. In embodiments where the second pore size is below 5 Å, for example 4 Å, gases with larger molecules (e.g., that have diameters of about 5 Å) are not captured. One example of a suitable zeolite for the first filter is the Sigma-Aldrich™ Molecular sieves, 4 Å beads, 1.6-2.6 mm with linear formula Na₁₂[(AlO₂)₁₂(SiO₂)₁₂]×H₂O.

The 3 Å and 4 Å are an effective combination to exclude water and retain some ethylene, whilst minimizing the collection of other potentially contaminating gases.

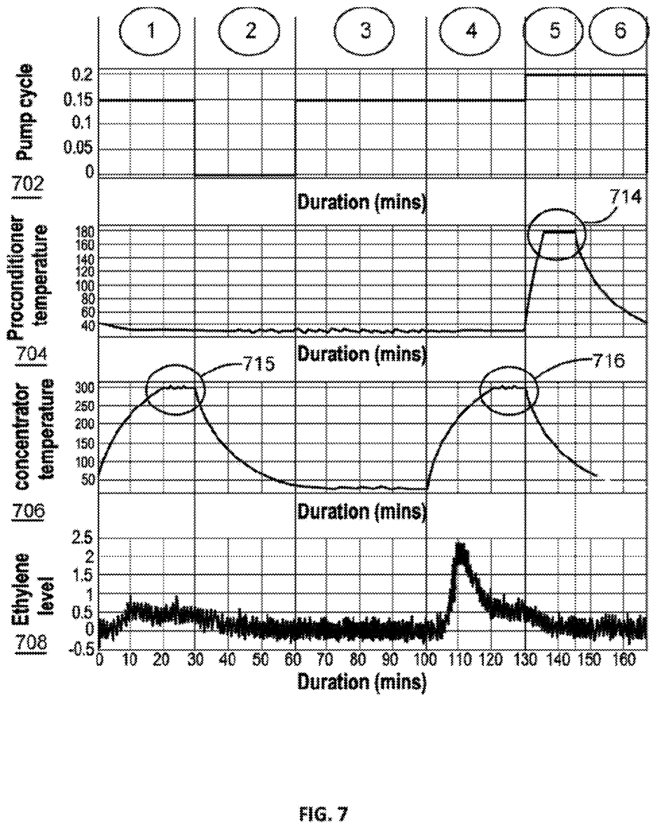

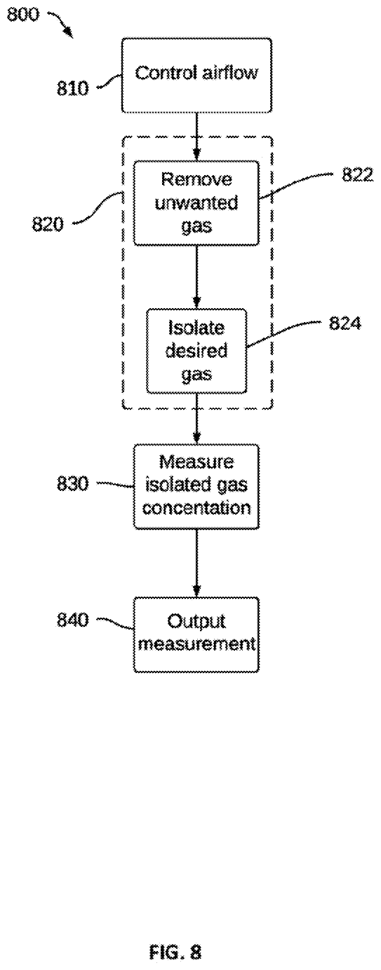

One of the characteristics of zeolites, is that heating a zeolite to a specific temperature results in the release of captured molecules. The inventors realised that the characteristics of zeolites can be used to both capture and subsequently release molecules, not only in order to remove some molecules, but also in order to retain certain molecules for measurement. With the aim of utilising a concentration means to increase the concentration of a gas component so that the component can be measured by a sensor, the inventors developed the innovative combination of more than one zeolite filter, together with both temperature control and airflow control over a measurement cycle, for example utilising a double heat cycle as described herein. This may be understood with reference to FIG. 7.

FIG. 7 of the drawings shows the target airflow 702, preconditioner temperature 704, concentrator temperature 706, and ethylene concentration 708 that define the operation of the system 100,400 over one sensing cycle (in this example lasting between 2 and 3 hours, and in the exemplary embodiment about 2.5 hours or about 170 minutes). With this type of cycle time, the systems described herein can advantageously provide multiple readings per day, enabling users to accurately track ethylene levels within the monitored environment. The sensing cycle includes six STAGES labelled 1 to 6 in FIG. 7, and this cycle is repeated continuously while the system is running, so that stage 1 follows stage 6.

The method includes a double heat cycle as seen in the concentrator temperature 706. The novel double heat cycle enables obtaining a baseline reading for the environmental (or carrier) gas which is then subtracted from the concentrated gas reading. In this way, if for example the air contains a contaminating gas, e.g., CO (that may, for example, result in triggering the ethylene sensor to show a 10 ppm reading, e.g., from 20 ppm of CO), then the first heat cycle will capture a baseline reading that measures the contamination plus accounts for the process of heating the gas up, and this first baseline reading is then subtracted from the reading of the second heat cycle. This novel double heat cycle removes some of the variables thereby enabling more accurate ethylene measurements. Furthermore, the double heat cycle rejuvenates the beads regularly, keeping them in a clean, reproducible absorbing state for each filtering cycle. The method therefore includes heating the concentrator to a high releasing temperature for a first heating cycle that releases a baseline gas for measuring a baseline, cooling the concentrator for a monitoring duration, and then heating the concentrating for a second heating cycle to release a monitored gas for measuring a target gas component. The method comprises determining a concentration of the target gas component by subtracting the measured baseline from the monitored gas.

The controller 112 is configured to control the filter and concentrator system 106 so that the first filter, in the illustrated embodiment the preconditioner 114, operates in a first state to capture an unwanted gas component, e.g. moisture in the form of water molecules. This occurs in stages 1 to 4. The controller 112 also controls the filter system so that the preconditioner 114 then operates in a second state to release the unwanted gas component. In this way, moisture and smaller gas molecules are removed from the air being monitored for ethylene. The system is configured so that any released unwanted gas components are released towards an exhaust and these unwanted gas components are expelled from the system. The peak 714 in the preconditioner temperature 704 in stage 5 is indicative of the second state, where the preconditioner is heated to a temperature between 150 and 200 degrees Celsius, for example about 180 degrees, thereby releasing the captured molecules towards the exhaust.

The controller 112 is configured to control the filter and concentrator system 106 so that the concentrator 116 operates in a first capture state to capture the target gas component (e.g. ethylene), as shown in stage 3 in FIG. 7. In stage 4 the concentrator 116 subsequently operates in a concentrating state (characterised by the temperature peak 716) to release the captured gas component, thereby isolating the gas component and creating a local increased concentration for sensing. The term “isolating” here means that the gas component is captured and retained, as opposed to filtered out, removed, lost, or captured and then released in a manner such that the gas component cannot be measured or sensed.

Isolating the gas entails amplifying and/or concentrating the gas for measurement by the sensor. Concentrating the target gas by isolating it as it passes through a concentrator that rejects unwanted gas components and sequesters the target gas, enables the concentration of the target gas components to increase to a concentration level that is measurable by the sensor.

During the stage 4 concentrating state, the target gas component is released toward the sensor system (for example toward a sensor chamber, or a sensor region of the system).

When the concentrator temperature is increased the concentrator releases the captured target gas in order to sense the concentration, but at the same time also performs a rejuvenating cycle that cleans out the zeolite beads in the relevant embodiments. Between each cycle, there are two rejuvenation cycles, one at heat peak 715 (stage 1) and one at heat peak 716 (stage 4) in the concentrator. In the example embodiment, the first part of these heat cycles (as the concentrator heat increases) is used for taking measurements with the sensor, while the second part of the heat cycles (at the sustained heat peak 715, 716) acts to rejuvenate the zeolite beads.

In some embodiments the filter and concentrator system operates like a molecular bandpass filter by capturing and removing molecules smaller than ethylene (such as water) in the first filtering stage, then allowing molecules larger than ethylene to move past the second concentrator stage, and thereby isolating and concentrating ethylene molecules in the second concentrator stage.

In the exemplary embodiments, the preconditioner 114 has a first associated heater 124 and the concentrator 116 has a second associated heater 122, so that the temperature of the preconditioner and the concentrator can be independently controlled, for example the preconditioner 114 may be heated while the concentrator 116 is cooled, and vice versa. The controller 112 activates the at least one heating element 120 to transition the concentrator 116 from the first capture state (that captures molecules) to the concentrating state (that releases molecules toward the sensor in a manner that increases the concentration of the molecules to a measurable concentration). The concentrating state, as can be seen in stage 4 in FIG. 7, is a heated state because the elevated temperature allows the captured molecules to be released from the zeolites of the concentrator.

The controller 112 operates the at least one heating element 120 so that the first state of the first filter occurs at a low temperature T1, for example at about an ambient temperature (under 40 degrees Celsius), and the second state of the first filter occurs at a first elevated temperature T2 (at about 200 degrees Celsius), causing release of any unwanted gas components (e.g. moisture, i.e. water molecules) from the first filter. The controller 112 also operates the at least one heating element 120 (which includes, in the illustrated embodiment in FIG. 1, the first preconditioner heater 124 and the second concentrator heater 122) so that the first capturing state of the concentrator occurs at a low (e.g. an ambient) temperature T3 (under about 40 degrees Celsius), and the second releasing state of the concentrator occurs at a second elevated temperature T4 (at about 300 degrees), thereby causing the release of the target gas component (e.g., ethylene), and thereby isolating the ethylene thereby increasing the concentration for sensing.

In some embodiments, T1 and T3 may be about the same, and/or T2 and T4 may be about the same.

Although changing the temperature in the preconditioner and concentrator aids in the adsorption and the release of difference gases, temperature changes alone are insufficient to manage how, where and when one or more gases can be sensed by the sensors.

The system 100,400 further comprises a pump 130,430, and the controller 112 is configured to operate the pump so that airflow from the gas input 102 to the gas output 104 via the filter and concentrator system 106 carries the isolated gas component (that has an increased concentration) to the sensor system 108 so that the sensor system senses the concentration of the isolated gas component. This can be seen in stages 3 and 4 in FIG. 7.

The controller 112 is also configured to operate the pump 130, 430 so that reverse airflow (which may be from the gas output 104 to the gas input 102, or which may be from alternative airflow routes) removes the unwanted gas component (e.g. moisture) without the unwanted gas contacting or affecting operation of the concentrator. This is occurs in stages 5 and 6, as illustrated in FIG. 7.

In some embodiments, the system may be operated to include stages 1 (illustrated in FIG. 7 of the drawings) in which an ethylene baseline is determined at the start of the ethylene monitoring cycle. This is done by increasing the temperature of the concentrator to about 300 degrees Celsius and simultaneously operating the pump to effect airflow from the gas inlet to the gas outlet, thereby releasing a baseline concentration of ethylene present in the concentrator towards the sensor before it has concentrated any gas. The system can then use the baseline conditions to subsequently determine the concentration of the ethylene more accurately.

In summary, the operation of the system as illustrated in FIG. 7 can be summarised according to the following six Stages:
(1) First Concentrator Heat Stage
(2) First Concentrator Cooldown Stage
(3) Saturation Stage
(4) Second Concentrator Heat Stage
Second Concentrator Cooldown that coincides with both:
(5) Preconditioner Heat Stage, and
(6) Preconditioner Cool Stage

FIG. 8 of the drawings illustrates one embodiment of a method 800 of monitoring a gas component such as ethylene. The method 800 comprises, at 810, controlling an airflow through a monitoring system, at 822, removing an unwanted gas component from the airflow (for example filtering out humidity by removing water molecules), and, at 824, concentrate to amplify the target gas component, e.g., ethylene, from the airflow. In some embodiments, steps 822 and 824 may be implemented by, at 820, applying a molecular bandpass filter to isolate the gas component. At 830 a sensor is used to measure the isolated gas component.

Removing the humidity from the airflow (822) includes first operating a first filter (e.g. in the form of a preconditioner) at a first temperature (e.g. at about an ambient temperature), and simultaneously operating a pump to cause airflow in a first direction (from a gas ingress to a gas egress), so that the water molecules are captured by the zeolites in the preconditioner. Subsequently, the preconditioner is operated at a second elevated temperature (e.g. about 200 degrees Celsius) and the pump is simultaneously operated to cause airflow in a second, reverse direction, so that the water molecules are released by the preconditioner without affecting operation of the sensor, because the humidity will not reach the sensor as the water molecules will be expelled from the ingress. The unwanted molecules are released toward an exhaust region of the system to dispel the unwanted molecules and to ensure that the unwanted molecules do not affect the measurements of the sensor system.

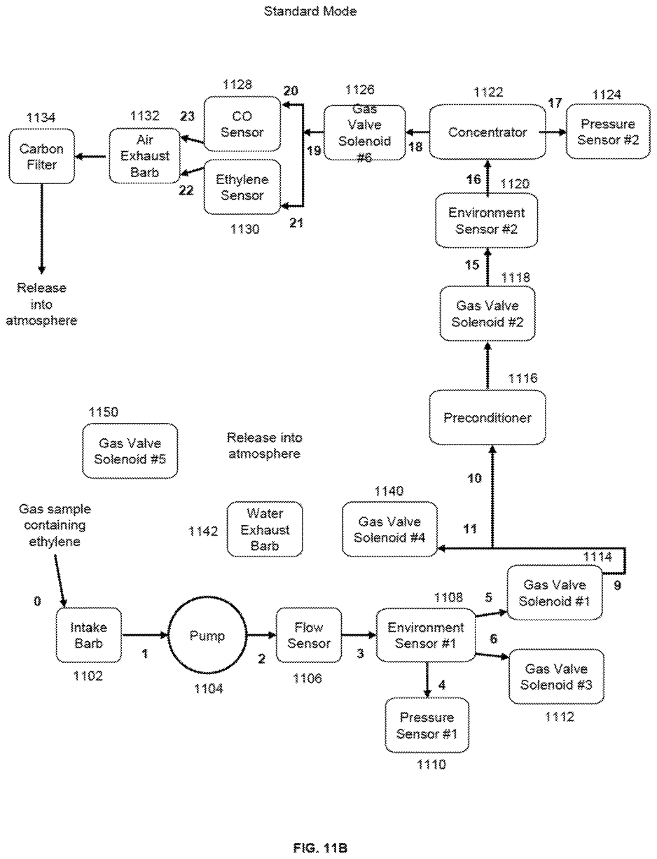

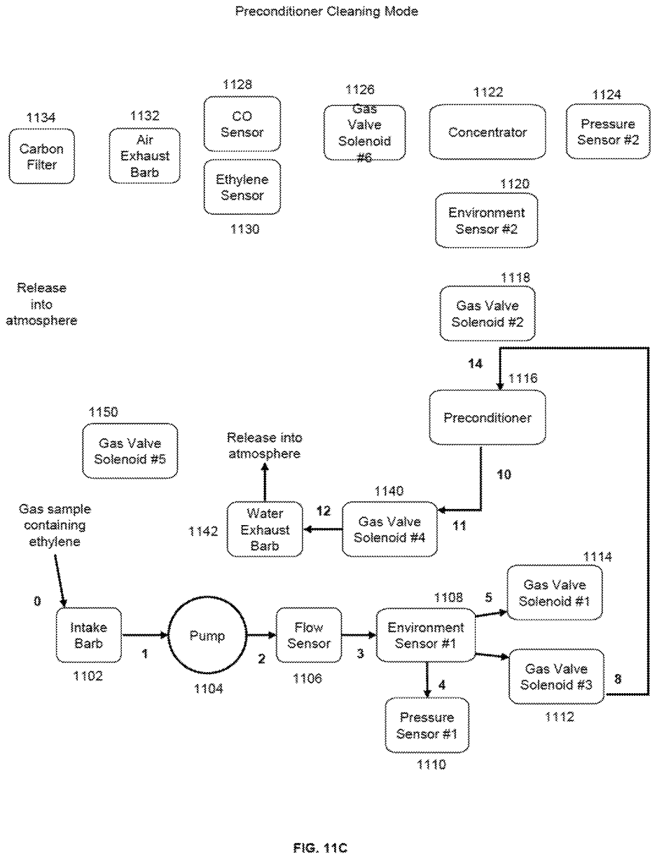

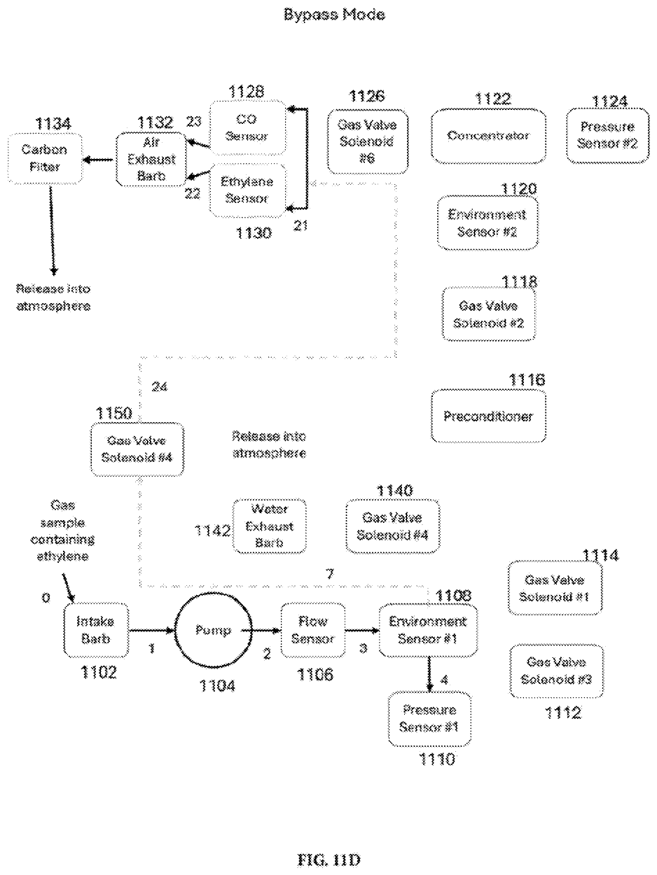

During the standard operating cycle (as described in more detail with reference to FIG. 11B), the air containing atmospheric water is pushed in through the bottom of the preconditioner chamber. This results in the accumulation of water in the preconditioner, as it is designed strip the water from the gas before the gas enters the concentrator.

The reverse flow cycle (as described in more detail with reference to FIG. 11C) is used to remove the captured water from the preconditioner during a cleaning cycle, and it ensures that water does not pass the tubing beyond the preconditioner tubing. Thus, by passing the air in reverse through the preconditioner, the water vapour is removed from the preconditioner. The water exits out of the system by bypassing the rest of the device circuitry which may be water sensitive. The exit opening for the reverse flow is physically positioned at the bottom of the system so that the water removal is assisted by gravity. Without the preconditioner and utilising the preconditioner cleaning cycle, the water would saturate the rest of the system, clogging up the tubing and interfering with the function of the concentrator and sensor if it were to be pushed forward through the system.

Isolating the gas component from the airflow (824) includes operating a concentrator at a first temperature (e.g. at about ambient, e.g. about 20-40 degrees Celsius) and simultaneously operating the pump to cause airflow in the first direction (from ingress to egress), so that the ethylene is captured by the zeolites of the concentrator. Subsequently the concentrator is operated at a second elevated temperature (e.g., at about 300 degrees Celsius) and the pump is operated simultaneously to continue airflow in the first direction, thereby isolating the ethylene and increasing the ethylene concentration to a measurable concentration. As a result, the concentration of the isolated ethylene can be measured at 830. At 840 the measured ethylene concentration is output for further processing and/or display on a user interface. In some embodiments, the method includes further processing of the data, for example determining a more accurate concentration of the ethylene based on the measured ethylene. This may be done by first measuring an ethylene baseline before isolating the ethylene for measurement, and then using the baseline to determine the accurate concentration of the ethylene (e.g., by a local or remote processor). Advantageously this double heat cycle permits a less than ideal sample gas to act as a carrier gas for the concentrated gas.

In some embodiments, measuring the gas component baseline comprises operating the concentrator at an elevated temperature (e.g. about 300 degrees Celsius) and simultaneously operating the pump to effect airflow in the first direction (from ingress to egress), thereby releasing a baseline of molecules (including ethylene and/or other impurities) present in the zeolites of the concentrator, also taking into consideration the impact of heating the concentrator. The baseline is taken into consideration by the system, in particular to calculate a more accurate ethylene measurement.

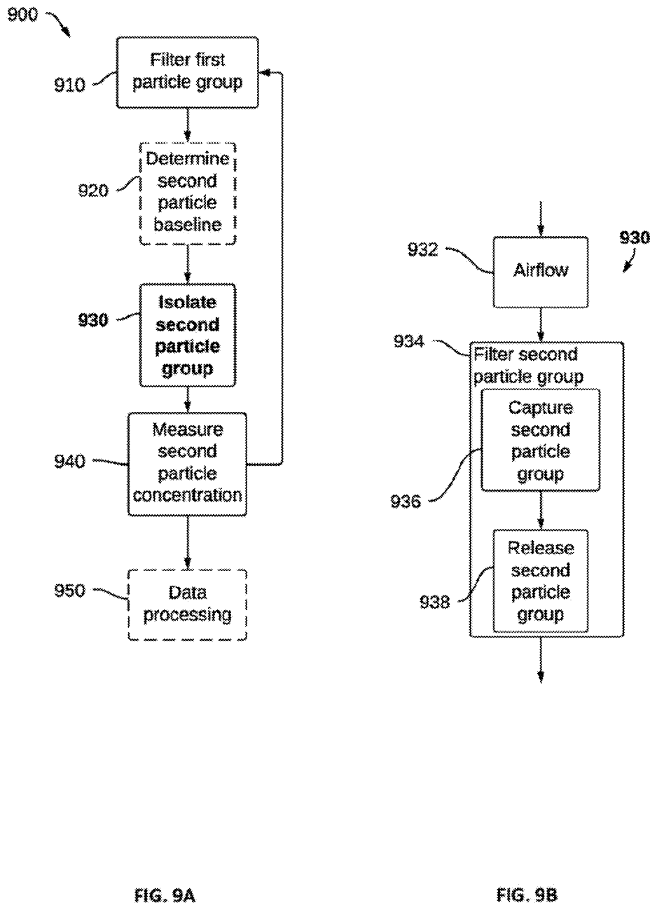

Operation of the systems described herein may also be understood with reference to FIGS. 9A and 9B. A method 900 of monitoring a second particle group (e.g. a group of particles that fall within a certain size range), comprises first, at 910, filtering out a first particle group (being in a different size range compared to the second particle group).

The method comprises at 930 concentrating to amplify the second particle group, and finally at 940 measuring the second particle concentration within the isolated second particle group. Optionally, the method 900 may include determining a second particle baseline before measuring the second particle concentration within the isolated second particle group. Optionally, the method 900 may include processing the measurement data (which includes the measured second particle concentration, and may also include the second particle baseline data).

As illustrated in FIG. 9B of the drawings, the step 930 of isolating the second particle group includes, at 932, maintaining an airflow (refer to the airflow 702 in stages 3 and 4 in FIG. 7) and at 934 filtering the second particle group from the airflow by capturing and releasing particles, thereby amplifying the second particle group. This is done by, first at 936 capturing the second particle group in the zeolites of the concentrator, and then at 938 releasing the second particle group by heating the zeolites of the concentrator. 938 also comprises rejuvenating the zeolites as part of the heating process.

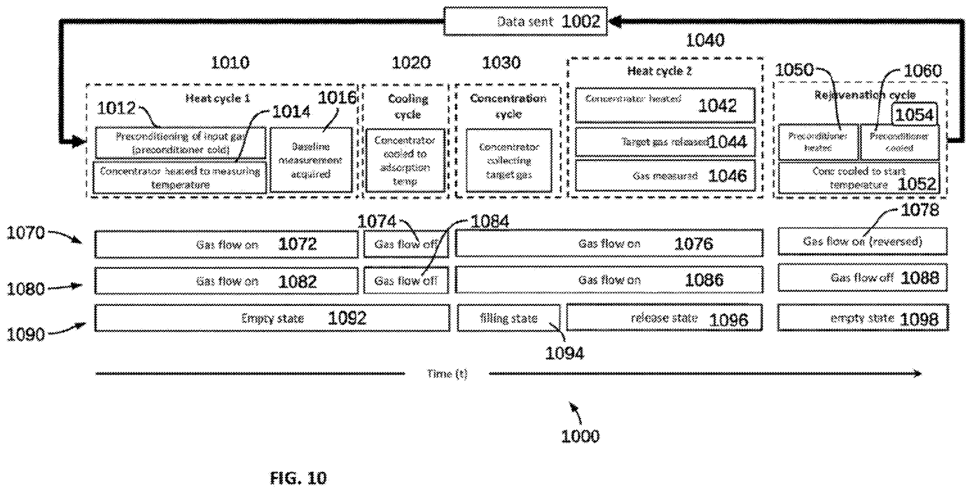

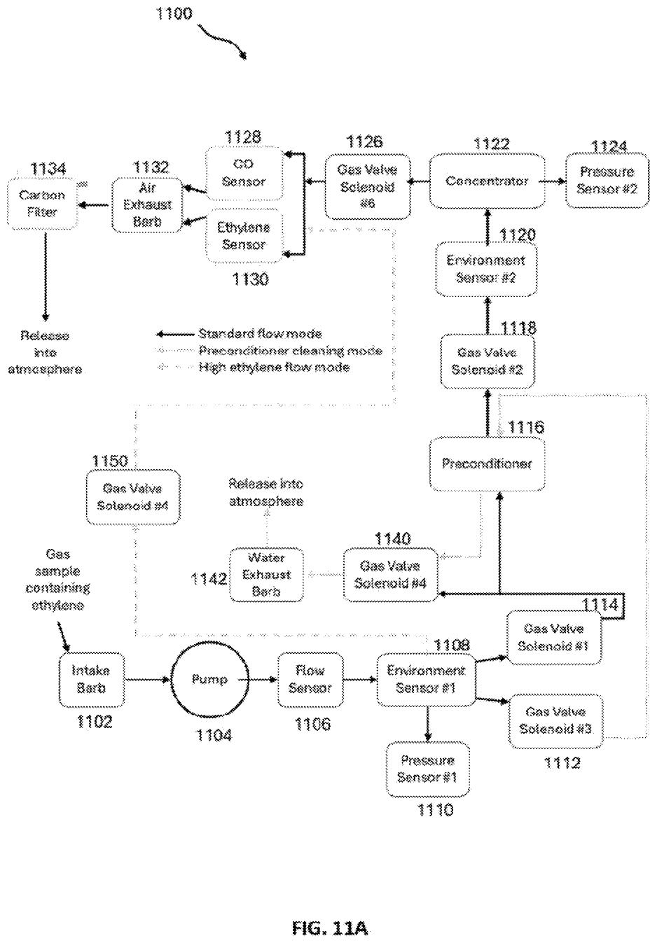

FIG. 10 of the drawings shows a timeline 1000 of parallel events during a measurement cycle performed by the systems described herein. As explained with reference to FIG. 7, the pump system (comprising one or more pumps and gas valve solenoids as described in more detail with reference to FIGS. 11A-11D), operates to effect airflow through the system and specifically through the preconditioner and the concentrator.

The preconditioner gas flow 1070 and the concentrator gas flow 1080 states are aligned with the six stages illustrated in FIG. 10 and similar to the six stages discussed with reference to FIG. 7 (in the schematic representation of FIG. 10, stages 5 and 6 are combined in the “Rejuvenation Cycle”).

Stage 1 (1010) is the first heat cycle in which the input gas is preconditioned at 1012 (the preconditioner is cold), and at 1014 the concentrator is heated to a rejuvenating temperature so that at 1016 the baseline measurement can be acquired. In embodiments 1016 occurs in parallel with 1012 and 1014, coinciding with heat cycle 2. During this first heat cycle 1010, the preconditioner gas flow is on at 1072, the concentrator gas flow is on at 1082, and the concentrator state 1090 is an empty state at 1092.

Stage 2 (1020) is the cooling cycle in which the concentrator is cooled to adsorption temperature. The preconditioner gas flow is off at 1074, the concentrator gas flow is off at 1084, and the concentrator gas state is still empty at 1092.

Stage 3 (1030) is the concentration cycle in which the concentrator collects the target gas, e.g., ethylene. During this stage the preconditioner gas flow is on at 1076, and the concentrator gas flow is on at 1086. The concentrator is in a filling state at 1094. The gas is amplified by the process of concentrating the gas, which results in a larger sensor reading. The amplification of the reading is related to (for example proportional to) the length of time of this saturation stage. In other words, the longer the concentrator is exposed the more ethylene will be stored in the concentrator. Similarly, an increased flow rate will also increase the amount of ethylene stored. During the concentration cycle, the concentration of the target gas component is increased from a small concentration (that may not be accurately measurable) to a larger measurable concentration that can be measured by a sensor, such as an electrochemical ethylene sensor.

In some embodiments the saturation time and/or flow rate may be adapted automatically based on the sensor reading(s) during one or more of the process stages. In other embodiments the saturation time and/or flow rate may be configured by a user via a system user interface, before and/or during operation of the system.

Stage 4 (1040) is the second heat cycle, in which the concentrator is heated to the rejuvenating temperature at 1042, the target gas is released at 1044, and the gas is measured at 1046. Steps 1042, 1044 and 1046 occur substantially in parallel, being substantially simultaneously as the temperature increases. Once the rejuvenating temperature has been reached (for example about 300 degrees Celsius), the zeolites are rejuvenated with the release of substantially all captured gas components.

The preconditioner gas flow remains on at 1076, and the concentrator gas flow also remains on at 1086. The concentrator state 1090 is a release state 1096 as the ethylene is being released. The forward gas flow (1086) pushes the released ethylene toward the sensor so that the gas is measured at 1046. If Stage 3 includes an increased time or flow rate, then the ethylene captured is amplified thereby enhancing the sensitivity of the sensor so that low concentrations of ethylene can be sensed.

Advantageously, the double heating method not only resets (or “rejuvenates”) the system thereby ensuring consistent operation, but also provides a baseline that captures any contaminating gases which can be removed via post-processing.

Stages 5 and 6 together form a rejuvenation or reset cycle 1054 that includes the preconditioner being heated at 1050 and the preconditioner being cooled at 1060. At the same time, the concentrator is cooled to the start temperature at 1052. During these two stages, in the preconditioner 1070 the gas flow is reversed at 1078, while the gas flow in the concentrator is off at 1088. The concentrator state 1090 is empty 1098 during the rejuvenation cycle 1054.

FIG. 11A of the drawings illustrates the overall system architecture 1100. Solenoid instructions are indicated in the green blocks, these instructions and resulting operation of the solenoids controlling the operational modes. The gas flow from one system component to the next is indicated for three operational modes: (1) normal flow mode for a standard measurement cycle (black/solid lines); (2) preconditioner cleaning mode for a preconditioner cleaning cycle (blue/dashed lines); and (3) high ethylene flow mode for a bypass cycle (red/dotted lines).

The architecture 1100 includes an intake barb, 1102, a pump 1104, a flow sensor 1106 a first environmental sensor 1108, and a first pressure sensor 1110. Following the first environmental sensor 1108 there are a first gas valve solenoid 1114 associated with the normal flow mode and a third gas valve solenoid 1112 associated with the preconditioner cleaning mode. The first gas valve solenoid 1114 is followed by a fourth gas valve solenoid 1140 and a water exhaust barb 1142, the latter two associated with the preconditioner cleaning mode. The architecture 1100 also includes a preconditioner 1116, a second gas valve solenoid 1118, a second environmental sensor 1120, a concentrator 1122 and a second pressure sensor 1124. For the normal flow mode, the architecture 1100 also includes a carbon monoxide sensor 1128, an ethylene sensor 1130 (e.g., a, Membrapor CA-10 sensor), an air exhaust barb 1132 and a carbon filter 1134. Associated with the high ethylene flow mode, the architecture 1100 includes a fifth gas valve solenoid 1150.

FIG. 11B illustrates the standard measurement cycle, where all the system components are in use except the water exhaust barb 1142 of the preconditioner cleaning cycle and the fifth gas valve solenoid 1150 of the high ethylene flow mode. This mode is used for low levels of ethylene (less than about 1-2 ppm), and both the preconditioner and the concentrator are in use.

FIG. 11C illustrates the preconditioner cleaning cycle in which the air is pumped in reverse flow to purge the preconditioner of water. In this mode the ethylene filled air enter the system via the intake barb 1102, and the pump 1104 pumps the air through the system via the flow, environment, and pressure sensors 1106, 1108, 1110. Following the gas valve solenoid 1112, the air passes through the preconditioner 1116 and via the fourth gas valve solenoid 1140 the humid air is released into the atmosphere at the water exhaust barb 1142.

FIG. 11D illustrates a bypass cycle that bypasses the preconditioner and concentrator in a high ethylene situation where the ethylene concentration is more than about 1-2 ppm. In this way, the concentration is first measured, and based on the first measurement the processor determines if the concentrator will be used or bypassed. If the concentration is above a threshold, for example 1 ppm, the processor directs the flow through to the sensor without concentrating it. This protects the concentrator from getting clogged up with too much ethylene.

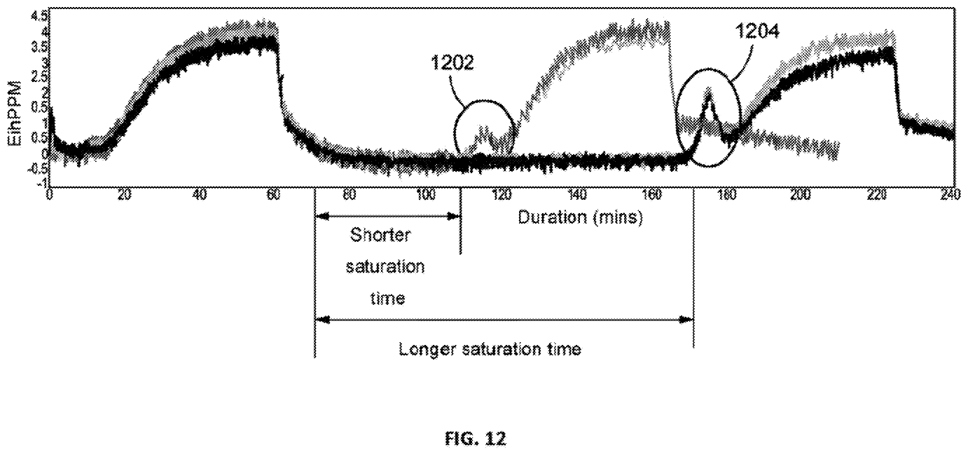

FIG. 12 shows ethylene sensor signals 1202, 1204 with respect to different cycle times. In some embodiments the cycle time is between 150 and 180 minutes, allowing the system to concentrate low levels of ethylene (e.g. 0.05 ppm) within the atmosphere. This amplifies the ethylene reading of the sensor system to within the accuracy range of the ethylene sensor. In other embodiments, the cycle time may be either increased or decreased to detect different levels of ethylene.

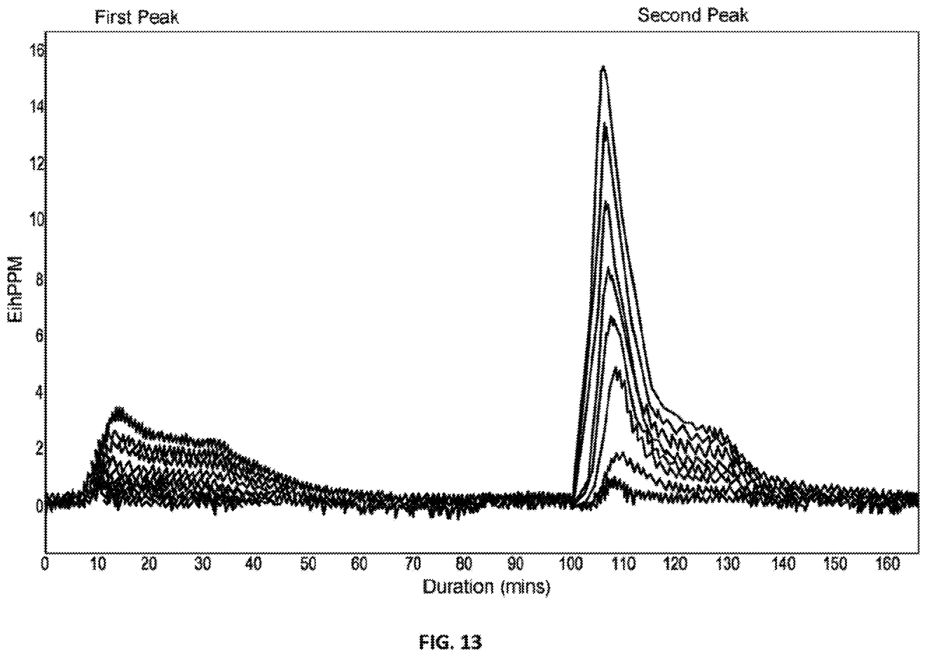

FIG. 13 shows ethylene sensor signals over the first and the second heat cycles, resulting in a first peak and a second peak. The double heat cycle occurs during a full measurement cycle, allowing the system to compensate for baseline conditions such as contaminating gases that are present in the air. The first peak of the heat curve is measured as soon as the cycle begins (while the concentrator is empty), and the second peak occurs after the saturation period.

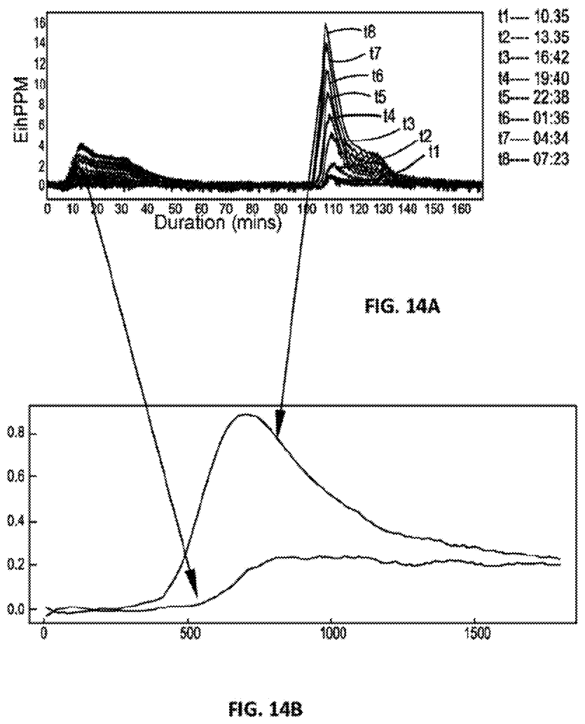

FIGS. 14A and 14B represent how the ethylene concentration is calculated from the initial reading illustrated in FIG. 14A. The first heat cycle results in the smaller peak on the left, and the second heat cycle results in the larger peak on the right. As illustrated in FIG. 14B the two readings from the first and second peaks are overlaid during post-processing of the sensor measurements, and then subtracted to give a more accurate ethylene concentration. A variety of different inputs may be used to determine the final ethylene concentration, including but not limited to the measured flow rates, humidity, temperature, and pressure (for example as described with reference to FIG. 5).

The algorithm automatically finds the overlapping (or closest overlapping points) between the two curves. This is advantageous as the curves may behave differently in various conditions, e.g. different temperatures. In some embodiments, the calculation may be performed by the controller within the system. In other embodiments, the measured data is communicated to a separate processor (for example by uploading the data to the cloud or sending the data to a remote server).

### Advantages and Benefits

Advantageously, the methods and systems described herein allow for the monitoring of very low concentrations of gas components, for example ethylene with a concentration of 1-2 ppm or less.

Advantageously, the ambient air is used as a carrier gas, with a baseline reading that represents the concentrator contents when it is heated and before the concentrator has been loaded with ethylene. Existing gas measuring devices typically use a clean carrier gas like nitrogen or helium (in a gas chromatography for example), which is costly and complicates the monitoring process.

When the gas is heated, it causes changes to the measured signal for a number of reasons, e.g., some of the ethylene could be pyrolyzed releasing CO, which could also (erroneously) trigger the ethylene sensor.

The benefit is provided by increasing the concentration of the gas component of interest to be a larger, measurable concentration that is measurable by a sensor. This method is both simpler and more cost effective compared to existing methods at the priority date for a number of reasons, including:
a. no specific or specialised carrier gas is required which means less consumables are required and various types of gas can be accommodated,
b. the air that is already present can function as a carrier gas,
c. the target gas component can be monitored irrespective of other components because the initial baseline assessment allows for this kind of simplified measurement technique,
d. the baseline is determined in the same sensing chamber where the gas component is measured, which results in a simpler and more cost effective set up,
e. no specialised operator is required as the system is simple to operate,
f. cost effective sensors can be used because the concentration of the gas component is increased so that typical sensors for that component can be used,
g. the method does not require any changes to air/gas pressure, resulting in a simpler and more cost effective solution,
h. compared to prior art gas chromatography systems, the systems and methods described herein are simpler and more cost effective,
i. the system does not require extreme temperatures such as subzero temperatures, making it cost effective and simple to implement,
j. by accumulating the target gas component by a set amount, the method allows standardising of measurements as well as control sensitivity,
k. nonpolar molecules such as ethylene can be measured,
l. the actual target gas component is measured, as opposed to some related indicator (i.e., a proxy) that provides a concentration estimate,
m. the target gas is not changed or processed in any way, but measured as is so that an accurate measure can be made of the target gas (and not of other molecules or gas components),
n. a simple configuration allows for measurements to be taken without a cooling requirements colder than an ambient temperature;
o. less sensitive sensors can be used because the gas is concentrated to levels that are easily detected by typical sensors, and
p. various gases can be used as a carrier gas, including a contaminated gas, because the method includes determining a baseline level of the target gas component.

### Example Embodiments

In one aspect there is provided a system for monitoring a gas component, the system comprising: a gas input in communication with a gas output via: a filter system for isolating the gas component from an airflow through the system; a sensor system adapted to sense a concentration of the isolated gas component; and a controller configured to control operation of the filter system.

The filter system may comprise a first filter for removing an unwanted gas component; and a second filter and/or a concentrator for isolating the gas component. The controller may be configured to control the filter system so that: the first filter operates in a first state to capture the unwanted gas component, and the first filter operates in a second state to release the unwanted gas component; and the second filter and/or a concentrator operates in a first filter state to capture the gas component, and the second filter and/or a concentrator operates in a second release state to release the filtered gas component, thereby isolating the gas component for sensing. The filter system may comprise at least one heating element, and the controller may activate the at least one heating element to transition the second filter and/or a concentrator from the first filter state to the second release state, wherein the second release state is a heated state.

At least one of the first filter and the second filter and/or a concentrator may comprise a molecular sieve. The first filter may comprise a molecular sieve with a first pore size, and the second filter and/or a concentrator may comprise a molecular sieve with a second pore size. The first pore size may be between 2 and 4 Å, and the second pore size may be between 3 and 5 Å. For example, the first pore size may be about 3 Å so as to remove water molecules from the airflow, and the second pore size may be about 4 Å so as to capture the gas component from the airflow.

The controller may operate the at least one heating element so that: the first state of the first filter occurs at about an ambient temperature, and the second state of the first filter occurs at a first elevated temperature causing release of the unwanted gas component from the first filter; and the first filter state of the second filter and/or a concentrator occurs at about an ambient temperature, and the second release state of the second filter and/or a concentrator occurs at a second elevated temperature causing release of the filtered gas component, thereby isolating the gas component for sensing.

The first filter may comprise a first aluminosilicate compound having a first pore size, and the second filter and/or a concentrator may comprise a second aluminosilicate compound comprising a second pore size, wherein the second pore size is larger than, the first pore size. Preferably, the second pore size is larger than and not overlapping with the first pore size.

The first aluminosilicate compound may comprise a zeolite with a pore size of about 3 Å, and the second aluminosilicate compound may comprise a zeolite with a pore size of about 4 Å.

The system may further comprise a pump, and the controller may be configured to operate the pump so that: airflow from the gas input to the gas output via the filter system carries the isolated gas component to the sensor system so that the sensor system senses the concentration of the isolated gas component, and reverse airflow from the gas output to the gas input removes the unwanted gas component without the unwanted gas contacting the second filter and/or a concentrator.

The filter system may operate as a molecular bandpass filter.

In another aspect there is provided a method of monitoring a gas component, the method comprising: controlling an airflow through a monitoring system; applying a molecular bandpass filter to isolate the gas component; and using a sensor to measure the isolated gas component.

Applying the molecular bandpass filter may comprise: removing an unwanted gas component from the airflow; and isolating the gas component from the airflow. Removing the unwanted gas component from the airflow may comprise: operating a first filter at a first temperature and simultaneously operating a pump to cause airflow in a first direction, so that the unwanted gas is captured by the first filter; and subsequently operating the first filter at a second elevated temperature and simultaneously operating the pump to cause airflow in a second direction, so that the unwanted gas is released by the first filter without affecting operation of the sensor.

Isolating the gas component from the airflow may comprise: operating a second filter and/or a concentrator at a second filter temperature and simultaneously operating a pump to cause airflow in a first direction, so that the gas component is captured by the first filter; and subsequently operating the second filter and/or a concentrator at a second elevated temperature and simultaneously operating the pump to continue airflow in the first direction, thereby isolating the gas and providing the isolated gas to the sensor for measurement.

The method may further comprise determining a concentration of the gas component based on the measured isolated gas component. The method may also further comprise measuring a gas component baseline before isolating the gas component; and using the baseline to determine the concentration of the gas component. Measuring the gas component baseline may comprise operating the second filter and/or a concentrator at the second elevated temperature and simultaneously operating the pump to effect airflow in the first direction, thereby releasing a baseline concentration of the gas component present in the second filter and/or a concentrator and providing the released baseline concentration of the gas component to the sensor for measurement.

The first filter, second filter, preconditioner and/or concentrator are not limited to aluminosilicate materials. More generally, any molecular sieve material or combination of materials having a microporous and/or mesoporous framework with a defined pore size distribution suitable for selectively adsorbing and/or excluding one or more target species (such as water, oxygen, nitrogen, argon, carbon dioxide, and/or volatile organic compounds) may be used. Suitable materials include, without limitation, aluminosilicate zeolites, silica-rich zeolites, aluminophosphate and silicoaluminophosphate frameworks (e.g. AlPO and SAPO materials), metal-organic frameworks (MOFs), covalent organic frameworks (COFs), carbon molecular sieves, activated carbons, porous polymer networks, and ordered mesoporous oxides such as mesoporous silica, alumina or titania, provided that they exhibit sufficient adsorption capacity, selectivity between the relevant molecular species, mechanical integrity, and regenerability under the operating conditions of the apparatus. In some embodiments, such molecular sieve materials are provided in the form of pellets, beads, granules, monoliths, coatings or structured bodies, and different molecular sieve types may be layered, mixed, graded or otherwise combined within a given filter or concentrator bed to achieve the desired overall separation performance.

In some embodiments, the first filter and/or preconditioner and the concentrator comprise one or more non-aluminosilicate molecular sieve materials having nominal pore sizes of about 3 Å and about 4 Å, respectively. For example, the first filter may comprise a metal-organic framework (MOF) having an effective pore aperture of about 3 Å, such as a zeolitic imidazolate framework (e.g. ZIF-7 or ZIF-11), configured to selectively admit or exclude molecules based on kinetic diameter in a manner analogous to a 3A zeolite. The concentrator may comprise a MOF having an effective pore aperture in the range of about 3.4-4.0 Å, such as a framework of the ZIF-8 or ZIF-67 type, or another ultramicroporous MOF designed to provide size- and/or shape-selective adsorption comparable to a 4A zeolite. In further embodiments, carbon molecular sieves or other ultramicroporous organic or inorganic frameworks with corresponding pore apertures may be used in place of, or in combination with, aluminosilicate zeolites, provided that they exhibit sufficient selectivity, capacity and regenerability under the operating conditions described herein.

It will be understood to persons skilled in the art of the invention that many modifications may be made without departing from the spirit and scope of the invention.

1. A method of measuring a small concentration of a target gas component in an environmental gas sample by increasing the small concentration to a larger measurable concentration, the method comprising:
isolating and concentrating, using a concentrator, the target gas component by capturing the target gas component and releasing the captured target gas component to a sensor, wherein isolating the target gas component comprises increasing a concentration of the target gas component so that the concentration of the target gas component is measurable by the sensor; and
measuring, using the sensor, the captured target gas component concentration,
wherein the method comprises,
before isolating the target gas component, heating the concentrator to release a baseline gas, and measuring, using the sensor, a baseline gas component concentration; and
after measuring the captured target gas component concentration, determining, with a processor, an accurate target gas component concentration by comparing the baseline gas component concentration and the measured target gas component concentration; and
wherein the method comprises removing, using a filter, an unwanted gas component, and wherein:
the filter comprises a first molecular sieve having a first pore size, and
the concentrator comprises a second molecular sieve comprising a second pore size larger than the first pore size.

2. The method of claim 1,
wherein removing, using a filter, the unwanted gas component comprises capturing the unwanted gas component and releasing the captured unwanted gas component to an exhaust;
and wherein the method comprises:
effecting a reverse airflow to remove the unwanted gas component without the unwanted gas contacting the concentrator; and
effecting a forward airflow to release the target gas component toward the sensor.

3. The method of claim 2, wherein capturing of the unwanted gas component and capturing of the target gas component occur at an ambient temperature, and wherein releasing and expelling the unwanted gas component and releasing and isolating the target gas component occur at elevated temperatures.

4. The method of claim 1, wherein the first molecular sieve comprises a first aluminosilicate compound having the first pore size, and the second molecular sieve comprises a second aluminosilicate compound comprising the second pore size larger than the first pore size.

5. The method of claim 1, wherein the filter and the concentrator operate together to cause a bandpass filtering effect that removes molecules smaller than a lower threshold size and larger than an upper threshold size from the sensor so that a concentration of molecules with a size between the lower threshold and upper threshold size is sensed by the sensor.

6. The method of claim 1, further comprising, before the isolating and concentrating, a bypass cycle comprising:
measuring, using the sensor, a concentration of the target gas component in the environmental gas sample and,
if the concentration is above a threshold, then directing airflow directly to the sensor without concentrating it,
so that the isolating and concentrating step occurs following and dependent on the bypass cycle.

7. A system for monitoring a gas component, the system comprising:
a gas input in communication with a gas output via:
a concentrator configured for capturing and then releasing the gas component from an airflow through the system; and
a sensor system configured to sense a concentration of the released gas component;
a controller configured to control operation of the concentrator to:
effect heating of the concentrator to release a baseline gas so that the sensor measures a baseline gas component concentration,
isolate the target gas component by capturing the target gas component and releasing the captured target gas component to the sensor system, thereby increasing a concentration of the target gas component so that the concentration of the target gas component is measurable by the sensor system;
a processor configured to determining an accurate target gas component concentration by comparing the baseline gas component concentration and the measured target gas component concentration, and
a first filter for removing an unwanted gas component, wherein the first filter comprises a first molecular sieve having a first pore size.

8. (canceled)

9. (canceled)

10. The system of claim 12, wherein the first aluminosilicate compound comprises a zeolite with a pore size of about 3 Å, and the second aluminosilicate compound comprises a zeolite with a pore size of about 4 Å.

11. The system of claim 7, wherein the concentrator is associated with a heater controlled to heat the concentrator during a baseline stage thereby causing the baseline gas to be released for sensing, and controller to heat the concentrator during a measurement stage thereby causing the release of the captured target gas component for sensing.

12. The system of claim 7, wherein the first molecular sieve comprises a first aluminosilicate compound having the first pore size, and wherein the second molecular sieve comprises a second aluminosilicate compound having the second pore size, wherein the second pore size is larger than the first pore size.
